(19) United States
(12) Patent Application Publication
(10) Pub. No.: US 2021/0121595 A1
(43) Pub. Date: Apr. 29, 2021

(54) TEXTILE PART

(71) Applicant: medi GmbH & Co. KG, Bayreuth (DE)

(72) Inventor: Patrick BAUER, Auerbach (DE)

(21) Appl. No.: 17/064,191

(22) Filed: Oct. 6, 2020

(30) Foreign Application Priority Data
Oct. 24, 2019 (EP) 19 204 990.6

(51) Int. Cl. A61L 15/22; A61F 5/01; A61F 13/08

(52) U.S. Cl. CPC A61L 15/22; A61L 2300/608; A61F 13/08; A61F 5/01

(57) ABSTRACT

A textile part, in particular a textile orthopedic aid, including a textile main panel as well as at least one functional element from plastics material which is disposed on the main panel, wherein the functional element is connected either in a form-fitting or a form-fitting and materially integral manner to at least one fastening element, and the fastening element is connected in a materially integral manner to the main panel.

### CROSS-REFERENCE TO RELATED APPLICATIONS

The present application claims priority of EP 19 204 990.6, filed Oct. 24, 2019, the priority of this application is hereby claimed and this application is incorporated herein by reference.

### BACKGROUND OF THE INVENTION

The invention relates to a textile part, in particular a textile orthopedic aid, comprising a textile main panel as well as at least one functional element from plastics material which is disposed on the main panel.

Such a textile part, as is known from EP 2 779 963 B1, for example, is embodied as a sleeve or as a bandage and serves for bearing on the body of a wearer. The textile part has a textile main panel on which at least one functional element from plastics material is disposed, said functional element being embodied as, for example, an elongate splint or as a joint or similar. This functional element serves for imparting to the textile part a specific functional property which is helpful to the wearer, thus acts in a stabilizing or reinforcing manner, for example, so as to protect in relation to mechanical influences and similar on that part of the body that is engaged across by the textile part.

Besides the possibility of incorporating such a functional element in a pocket which is preferably configured on the main panel by way of knitting technology, thus for example for pushing a stabilizing rod into a corresponding elongate pocket, for example, it is also known for the functional element to be fixed to the main panel by way of a materially integral connection, thus a welded connection. This is known, for example, from EP 2 779 963 B1 where a functional element is welded to the main panel, for example a bandage, by way of a carrier element which is laminated with plastics material. The functional element per se is applied to the carrier element, this taking place for example by over-molding or back-molding, in a first process step. This combined piece part in a second process step is thereafter placed on the bandage and welded exclusively in the region of the carrier element, the latter being wider than the functional element, this taking place by fusing the plastics-material coating of the carrier element. The functional element per se in this design embodiment is not connected to the main panel but connected to the latter merely by way of the carrier element, this leading to potential deficiencies arising in the functionality of the functional element since the properties of the latter, for example stabilizing properties, are not transmitted directly to the main panel. Moreover, the production procedure is also complex since the combined piece part composed of the functional element and the carrier element has initially to be produced in a first process step, whereupon the application of this combined piece part to the main panel takes place only in a second process step.

### SUMMARY OF THE INVENTION

The invention is thus based on the object of specifying a textile part which is improved in comparison to the above.

In order for the object to be achieved it is provided according to the invention in a textile part of the type mentioned at the outset that the functional element is connected either in a form-fitting or a form-fitting and materially integral manner to at least one fastening element, and the fastening element is connected in a materially integral manner to the main panel.

As opposed to the prior art, the functional element and the fastening element in the textile part according to the invention are in any case connected to one another in a form-fitting manner, that is to say that corresponding form-fit geometries or contours which engage in one another, or interact with one another, respectively, and thus enable a form-fitting disposal of the functional element on the fastening element, are provided on the functional element and on the fastening element. In one alternative of the invention there is additionally the possibility of also providing a materially integral connection between the functional element and the fastening element, that is to say that the fastening element and the functional element in this case are connected to one another in a form-fitting manner as well as in a materially integral manner, to which end corresponding materials which are able to be connected in a materially integral manner to one another are provided on the functional element as well as on the fastening element. In the case of the functional element being composed of plastics material, this can be the plastics material per se, in the case of the fastening element this can be, for example, a corresponding plastics-material coating or any other content of plastics material provided proximal to the fastening element, such that the two elements can be connected to one another in a materially integral manner in the context of a plastics welding method or optionally also in the context of a plastic coating method.

If only one type of connection, thus the form-fitting connection, is provided, a sufficiently stable connection between the functional element and the fastening element which is fastened in a materially integral manner to the main panel can already be established on account thereof, said main panel preferably potentially being a textile knitted panel, preferably having compressive properties. This variant moreover also has the advantage that, depending on the design embodiment of the connection, there is also the possibility of releasing the functional element from the fastening element, consequently for the latter to be removed from the form-fitting connection and, when required, to be replaced by another functional element which of course has to have an identical or comparable, respectively, geometry at least in the region of the form-fitting connection. In the case of a textile part having a functional element in the form of a stabilizing splint, this enables a correspondingly conceived stabilizing splint to be fixed as a functional element on the fastening element when required, for example depending on the desired degree of stiffness. This means that the textile part, optionally also according to a therapeutic prescription, can be specified by choosing a corresponding functional element which in terms of the mechanical or functional, respectively, properties thereof is correspondingly conceived.

Since the functional element is a plastics-material element, the corresponding functional or mechanical, respectively, properties can also be indicated by way of corresponding coloring. For example, a functional element dyed green, for example in the form of a stabilizing stiffener, may have only a relatively minor stiffness, while a functional element dyed red is stiffer in comparison, up to a stabilizing element which is dyed blue, for example, and which is the stiffest functional element within the range.

If a form-fit as well as a materially integral fit is used, a particularly strong connection between the functional element and the fastening element is provided, said particularly strong connection by virtue of the form-fitting connection enabling the functional properties of the functional element to be transmitted in an optimal manner to the main panel by way of the fastening element. This is because the support, or the immobilization effect, respectively, resulting from the stiffness of the functional element, for example in the form of a stabilizing stiffener, as a result of the form-fitting connection in addition to the materially integral connection, can be transmitted to the main panel even better than is the case with only a materially integral connection as known in a simple manner from EP 2 779 963 B1, for example, where the functional element in the form of a planar stabilizing stiffener only bears in a planar manner on the carrier element but per se is not coupled in either a materially integral or form-fitting manner.

In an embodiment of the combination of elements composed of the functional element and the fastening element using a form-fitting and materially integral connection, there is moreover the possibility of fixing this unit on the main panel in a single process step. To this end, it is only necessary for the functional element and the fastening element to be initially connected to one another in a form-fitting manner and to be placed on the main panel, either on the external side or the internal side of the latter. The actual welding of the piece parts to one another is then performed by an input of energy, for example in the form of high-frequency or ultrasonic vibrations. Local fusing or softening, respectively, of the plastics material of the functional element arises on account of this input of energy, on the one hand, such that a materially integral connection upon cooling takes place in the portions in which said functional element bears on the fastening element, thus also in the region of the form-fit. In the context of this input of energy, softening or fusing of the content of plastics material in the functional element also arises, on the other hand, at least in those regions in which the fastening element bears on the main panel, such that wetting of the main panel, thus the textile structure thereof, arises also there by virtue of the softening or fusing, respectively, of this plastics material, the fused plastics material connecting in a materially integral manner to the main panel, optionally also slightly penetrating the textile structure of the latter. This means that a materially integral connection, in addition to the form-fit provided, between the individual piece parts can be achieved in the textile part according to the invention in the context of a single process step by a corresponding input of energy for heating and fusing the corresponding material regions.

While the materially integral connection is preferably established by corresponding welding, thus by plastics welding of the piece parts, in that the corresponding input of energy for fusing is in particular performed by radiation energy, it is however likewise conceivable for the fusing energy to be applied in another manner, for example in a purely thermal manner by incorporating said fusing energy in a heating oven or the like.

The fastening element and the main panel are in any case connected to one another in a materially integral manner, thus welded, as described. Additionally, a form-fitting connection may also be provided, for example in that said fastening element and said main panel are sewn to one another, wherein the thread establishes the form fit here. Riveting is also conceivable.

The fastening element per se can be either flexible or rigid. “Flexible” is understood to be a corresponding flexibility of the fastening element, this enabling that the fastening element can fundamentally follow corresponding movements of the main panel, or corresponding variations in the geometry of the main panel, respectively, to a sufficient extent, thus for example when the main panel adapts to a region of a body located below said main panel, such as a lower leg or similar, and said fastening element can readily conjointly perform said adaption. The fastening element is thus far more flexible than the functional element, in this instance in the form of a stabilizing stiffener or the like, for example. The fastening element in such a case can be of a film-type, so to speak, having a corresponding wall thickness of, for example, 0.5 mm to 1.5 mm, wherein correspondingly greater wall thicknesses can be provided in the region of the corresponding form-fit geometries, for example, so as to guarantee a corresponding mounting of the form-fit. This can also be a textile carrier which has a plastics-material coating and which is likewise very flexible. In contrast, “rigid” is understood to be a mechanical property of the fastening element which is at least very similar or close to the mechanical properties of the functional element. This means that the fastening element per se has a sufficient stiffness and in itself already sufficiently stiffens the main panel, the latter preferably being a textile knitted panel, as described. Here too, the fastening element can be from plastics material, but in this instance either has a correspondingly greater thickness, for example in the range from 1.5 mm to 2.5 mm, or is made from a correspondingly stiff, or less flexible, respectively, plastics material. This means that a therapeutic purpose which is to be met by the textile part or the functional element, respectively, can fundamentally be taken into account also by the choice of the design embodiment of the fastening element, since the fastening element can be conceived so as to facilitate said purpose.

As described, there is the possibility for the fastening element, like the functional element, to be a plastics-material element. Alternatively, the fastening element can also have a carrier having a plastics-material coating on one side or both sides. The carrier can be, for example, a textile, for example a velour, optionally also having hook-and-loop properties, that is to say that elements can be additionally fastened to said carrier by way of a hook-and-loop connection, this even further enhancing the functionality of the unit composed of the functional element and the fastening element. A velour, or a hook-and-loop velour, respectively, is a medium-fine warp-knitted material in which individual filaments of thread floats by way of a finishing process such as raising, in particular on a roller raising machine, are lifted and solidified so as to form loops in order to be hook-and-loop capable. The invention is however not limited to the use of such a velour; rather, each textile, or each textile layer, respectively, in the form of a woven fabric, a warp-knitted fabric, or a circular or flat knitted fabric, can be used as a fastening element. Depending on whether a materially integral connection is to be performed only toward the main panel or also toward the functional element, this carrier is provided with a plastics-material coating either on one side, only on the lower side of said carrier that faces the main panel, or on both sides, thus also at least in those regions in which a materially integral connection toward the functional element is to be established. This plastics-material coating can be laminated onto the carrier, thus the textile carrier, for example, or else be sprayed onto said carrier.

Besides the fastening element per se being equipped with the plastics-material coating, it is however also conceivable for the fusible plastics-material plane to be established by interposing between the fastening element and the main panel a corresponding intermediate tier which establishes the materially integral connection, that is to say that a corresponding planar, fusible plastics-material film or similar is placed therebetween, said plastics-material film then being correspondingly fused by way of the input of energy such that the fastening element, in this instance the remaining carrier of the latter, as well as the main panel are connected in a materially integral manner to the fused intermediate tier.

If the materially integral connection is established by welding, thus plastics welding, the corresponding materials of the plastics-material elements which are to be connected to one another herewith, thus of the functional element and of the fastening element when the latter are to be connected to one another in a materially integral manner, are of course to be chosen such that said materials can connect in a materially integral manner, consequently are chemically compatible to this extent. In principle, the functional element and/or the fastening element, or the plastics-material coating, or the intermediate tier can be composed of polyamide, polyvinylchloride, polyurethane, polyethylene, or a thermoplastic elastomer, in particular a urethane-based thermoplastic elastomer. This list is however not exhaustive; further plastics-materials which have corresponding properties either in terms of welding or in terms of displaying the corresponding mechanical properties, can also be readily used.

In principle, there is the possibility of disposing only one functional element on a fastening element, thus only one stabilizing splint or similar, for example. Depending on the type of the textile part and/or depending on the type of the therapeutic task, it can however also be necessary to dispose of a plurality of functional elements either in a form-fitting manner or form-fitting and materially integral manner on a common fastening element. For example, two or more stabilizing splints can be disposed beside one another, said stabilizing splints potentially running in a mutually parallel manner or at a mutual angle, or said stabilizing splints having different shapes or lengths, etc. 1a This means that a variation potential for adapting to a therapeutic task in the best possible manner exists to a relatively large extent by way of the latter. It is also conceivable herein for the fastening manner of the plurality of functional elements to be designed in dissimilar ways. For example, a central functional element which is to be mandatorily disposed can be disposed in a form-fitting and materially integral manner, thus not be replaceable, while one further functional element or a plurality of further functional elements are attached only in a form-fitting manner, for example, such that said further functional elements can be retrieved or replaced by others when required, etc.

It is furthermore conceivable that a functional element which is composed of a plurality of parts that are able to be moved relative to one another is provided, wherein one part is connected either in a form-fitting or a form-fitting and materially integral manner to a first fastening element, and a further part is connected either in a form-fitting or a form-fitting and materially integral manner to a second fastening element, and both fastening elements are fastened in a materially integral manner to the main panel. In this design embodiment, a joint-type functional element which has two articulated legs which by way of an articulated connection are pivotably connected to one another is used as a functional element. Each articulated leg is fixed on, or in, respectively, a separate fastening element, wherein both fastening elements are fastened in a materially integral manner on the main panel. The fixing of the articulated legs to the fastening elements here can also only be in a form-fitting manner such that the joint can also be replaced when required, for example a tight joint being replaced by a looser joint, or a joint permitting only a small pivot angle being replaced by a joint permitting a larger pivot angle. Alternatively, a materially integral connection may of course also be provided. In principle, there is the possibility here too for a further functional element such as, for example, in this instance a relatively short stabilizing stiffener or similar, to be disposed in addition to the joint functional element on the first and/or the second fastening element in both alternative fastening variants.

A central feature of the invention is the form-fitting fastening of the form-fitting element on the fastening element. To this end, at least one groove which is undercut on one side, or a groove undercut on both sides, can be provided on the fastening element. In the case of a groove undercut on one side, the functional element has a corresponding form-fit geometry which is compatible in terms of shape, said functional element thus having a corresponding rebate or groove on one side such that corresponding engagement is possible. The form-fit element in this case thus has an L-shape or C-shape, so to speak. In the case of a groove undercut on one side, a straight groove leg can be provided on the side that is opposite the undercut groove leg; alternatively, the functional element here may also be exposed. In this case, the functional element would then expediently also have to be fixed in a materially integral manner.

In the case of a groove undercut on both sides, the functional element is fixed in a form-fitting manner on both sides and has a T shape having rebates on both sides, or a double-T shape with grooves on both sides for engagement. A materially integral connection is not necessarily required here but may be provided as described.

Alternatively, it is also conceivable for at least one T-shaped protrusion which is received in a groove undercut on both sides on the functional element to be configured on the fastening element. The fastening element here is snap-fitted in the groove proximal to the functional element, so to speak, the materially integral connection being established herewith. Alternatively, an L-shaped protrusion which engages in a groove undercut only on one side on the functional element can also be configured on the fastening element. A corresponding form-fitting connection can also be implemented herewith.

In principle, the form-fitting connections are preferably always distinguished by a corresponding engagement in or across said functional element, the fixing taking place therewith.

In a refinement of the invention it is expedient for one through-hole or a plurality of through holes to be provided on the fastening element. The bearing face of the fastening element on the main panel is reduced on account of said through holes, that is to say that the fastening face and thus also the connection plane by way of which the fastening element is connected in a materially integral manner to the main panel is reduced. The main panel, thus for example the textile knitted panel, on account thereof remains somewhat more flexible, this being in particular expedient in the case of a more flexible fastening element which is after all specifically intended to conjointly perform any geometrical variations of the main panel.

While there is the possibility for using the individual piece parts, thus the functional element, the fastening element, and the main panel, as separate elements and to fix said piece parts to one another in the preferably single-stage method, it is however also conceivable in one refinement of the invention for the functional element or functional elements and the fastening element or fastening elements to be joined in a plastic coating method, in particular a two-component plastic coating method. The functional element which is composed of plastics material anyway, and the fastening element, irrespective of whether the latter is a fully plastic component or has a carrier with a plastics-material coating, can always be joined in a plastic coating method, that is to say that both are sprayed simultaneously and joined herein, thus coupled to one another in a form-fitting manner. It is also conceivable that the functional element in a plastic coating method is sprayed onto the carrier having the plastic coating and is thus joined in a form-fitting manner to said carrier.

The main panel per se is a knitted panel, as described, wherein a compressive knitted panel is in particular used. A compressive knitted panel exerts a defined compression, thus force, on the part of the body, or the tissue, respectively, lying therebelow and engaged across by said knitted panel. In terms of such a compressive textile part in the form of medical compression stockings reference is made in a purely exemplary manner to the definitions in “Quality Assurance RAL-GZ387/1 Medical Compression Stockings” and in terms of medical compression arm hosiery to “Quality Assurance RAL-GZ387/2 Medical Compression Arm Hosiery” of Gütezeichengemeinschaft medizinische Kompressionsstrümpfe e. V (Quality Seal Association of Medical Compression Hosiery), where corresponding quality and testing guidelines are stated for medical compression stockings (and arm hosiery) which can likewise be a textile part according to the invention.

The textile part per se can be one of various types. This can be a bandage, an orthotic, or a stocking, in particular compression stocking, on which an elongate or curved rod-type functional element, a joint-type functional element, a functional element configured as a belt loop, a functional element configured as a traction aid which is able to be manually gripped, or a functional element configured as a pad is provided. The most varied variation possibilities in terms of the design embodiment of the textile part and of the therapeutic purpose of the latter are thus provided here, just like the most varied functional elements can also be used.

Besides the textile part per se, the invention furthermore relates to a method for producing such a textile part. Said method is distinguished in that at least one functional element which is connected either in a form-fitting or a form-fitting and materially integral manner to at least one fastening element is disposed on a textile main panel, whereupon a materially integral connection between the fastening element and the main panel is established by an input of energy.

Moreover, a materially integral connection between the functional element and the fastening element can also be established by the input of energy, that is to say that not only the form-fitting connection but additionally also a materially integral bond is generated there.

Establishing the materially integral connection preferably takes place according to the invention in that the fastening element, or the fastening element and the functional element, or an intermediate tier disposed between the fastening element and the main panel, is/are locally fused in order for the connection to be established. The input of energy preferably takes place by radiation energy, in particular by high-frequency or ultrasonic vibrations, that is to say that a plastics welding method is ultimately used in order for the materially integral bond to be established, since the corresponding fusible piece parts are all made of plastics material or at least have a fusible content of plastics material.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows a schematic illustration in the form of an exploded view of a textile part according to the invention, having a separate functional element, a separate fastening element having a separate main panel;

FIG. 2 shows the assembly from FIG. 1 having a fastening element placed on the main panel, having a functional element already fixed thereto in a form-fitting manner;

FIG. 3 shows the assembly from FIG. 2 in the welded final state;

FIG. 4 shows a schematic illustration of a second embodiment of a textile part according to the invention in a partial view;

FIG. 5 shows a schematic illustration of a third embodiment of a textile part according to the invention in a partial view;

FIG. 6 shows a schematic illustration of a fourth embodiment of a textile part according to the invention in a partial view;

FIG. 7 shows a schematic illustration of a first specific form of implementation of a textile part in the form of a bandage;

FIG. 8 shows a schematic illustration of a second specific form of implementation of a textile part in the form of an orthotic;

FIG. 9 shows a schematic illustration of a third specific form of implementation of a textile part in the form of a stocking; and

FIG. 10 shows a fourth specific design embodiment of a textile part in the form of a stocking having a belt loop.

### DETAILED DESCRIPTION OF THE INVENTION

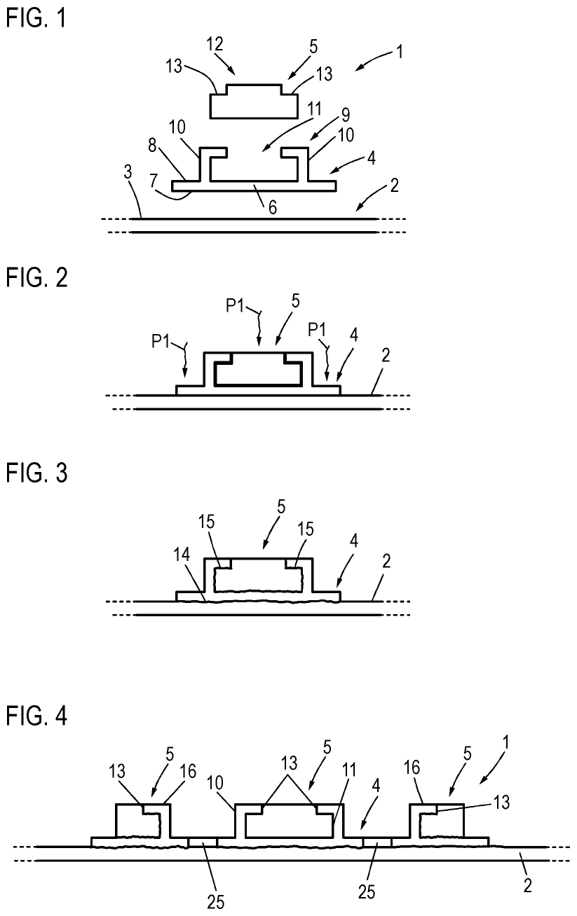

FIG. 1 in the form of an exploded view shows a textile part 1 according to the invention, comprising a main panel 2, preferably a textile knitted panel, in particular having compressive properties. The main panel 2 has an external side 3 onto which a fastening element 4 as well as a functional element 5, which is fixed to the fastening element 4, are subsequently placed. Alternatively to the disposal on the external side 3, there is of course also the possibility of placing the functional element 5 and the fastening element 4 on the opposite internal side of the main panel 2.

The fastening element 2 presently is a plastics-material element, for example, in the manner of an elongate, comparatively flexible or rather comparatively stiff plastics-material film, for example, which has a corresponding element base 6 having a lower side 7, wherein a form-fit contour 9 is configured on the opposite upper side 8 in the example shown, said form-fit contour 9 comprising two legs 10 which define a groove 11 which is undercut on both sides and into which the functional element 5, embodied so as to be compatible in terms of shape, can be inserted, or snap-fitted, respectively.

To this end, the functional element 5 which is likewise composed of plastics material likewise has a corresponding form-fit contour 12 on both sides, said form-fit contour 12 comprising two rebates 13, the transversely running portions of the legs 10 in the snap-fit position engaging into said rebates 13, see FIG. 2.

The functional element 5, for example a stabilizing splint, is likewise from plastics material, as described. This herein can be the same plastics material as that of the fastening element 4, or can be another plastics material, as long as said functional element 5 and said fastening element 4 can both be connected in a materially integral manner to one another by a plastics welding method, should this be desired. However, a materially integral bond is not mandatory since a form-fitting bond is provided.

The fastening element 4 and the functional element 5 can also be of identical color; alternatively, it is however also conceivable for the functional element 5 to have a different color than the fastening element. In the case of a black main panel 2, thus a black textile knitted panel, for example, the fastening element is also embodied so as to be black in order for said fastening element to be as inconspicuous as possible. In contrast, the functional element 5 can have a significantly different color, wherein this other color can be chosen for design reasons, on the one hand, or else in order to indicate herewith which mechanical properties the functional element 5 has, thus for example how stiff the latter is, on the other hand, if the functional element 5 is a stabilizing stiffener.

In the next step shown in FIG. 2, the functional element 5 is first snap-fitted or latch-fitted into the groove 11 of the fastening element 4 such that said functional element 5 is fixed and received in a form-fitting manner in said groove 11. A reliable fastening is provided by way of this form-fit, just like it is also ensured herewith that the mechanical properties, thus the functional properties of the functional element 5, are mandatorily transmitted to the fastening element 4. Since the fastening element 4 is fixedly connected to the main panel 2, as will yet be discussed hereunder, these properties in this instance are herewith also mandatorily transmitted to the main panel 2 and thus also to the part of the body which is engaged across by said main panel 2.

It can be seen in FIG. 2 that the fastening element 4 by way of the lower side 7 bears on the external side 3 of the main panel 2, thus providing contact across a large area.

While the fastening element 4 and the functional element 5 in FIGS. 1 and 2 are shown as separate piece parts, it is to be noted that said fastening element 4 and said functional element 5 can already be produced as a two-component piece part by a two-component injection-molding method at the factory or the production facility such that both piece parts after the injection-molding are already connected in a form-fitting manner to one another. Separate joining, as shown in FIGS. 1 and 2, is not required in this instance; rather, the two-component piece part composed of the fastening element and the functional element can be placed directly on the main panel and subsequently be welded, this being discussed hereunder in the next step which is likewise shown in FIG. 2.

In the next step, likewise illustrated in FIG. 2, a materially integral connection at least between the fastening element 4 and the main panel 2, or else between the functional element 5 and the fastening element 4 in the example shown, is now established, this however not being mandatory since the functional element 5 by way of the form-fit is already firmly fixed to the fastening element 4. In order for the materially integral welded connection to be established, an input of energy takes place in a single process step, as is illustrated by the arrows P1. The energy is preferably supplied in the form of high-frequency or ultrasonic vibrations, plastics welding thus being performed. The input of energy has the effect that local fusing of the fastening element on the surface takes place in the region where said fastening element bears on the main panel, thus in the region of the lower side 7 of said fastening element. On account thereof, wetting of the surface of the main panel 2 arises on account of the fused plastics material, the latter optionally also somewhat penetrating the textile structure of the main panel 2 such that a fixed, materially integral bond arises after the fused plastics material has cured.

In the example shown, a materially integral connection between the functional element 5 and the fastening element 4 is implemented in the region of the form-fitting connection. This means that fusing of the material of the fastening element 4 as well as of the functional element 5 takes place in the respective neighboring boundary region on account of the input of energy such that a materially integral connection is also provided there upon curing.

The completed fixed final state is shown in FIG. 3. It can be seen that a materially integral connection 14 is provided between the fastening element 4 and the main panel 2, as is illustrated by the serrated lines, as is also a materially integral connection 15 between the functional element 5 and the fastening element 4 in the region of the groove, this likewise being illustrated by the serrated lines.

FIG. 4 shows a further alternative of a textile part 1 according to the invention, wherein the same reference signs are used for identical elements. Provided again are a fastening element 4 as well as three functional elements 5 and one main panel 2 in the example shown, said main panel 2 again preferably being a textile knitted panel. Three different fastening points are presently provided on the fastening element 4, specifically a centric fastening point which corresponds to that from FIGS. 1 to 3 and is implemented by way of the two L-shaped legs 10 which define the groove 11 undercut on both sides. The functional element which has the two rebates 13 is already snap-fitted into said groove 11 in a form-fitting manner; this form-fitting connection here is final, that is to say that no materially integral connection is provided in this exemplary case.

Furthermore provided are two legs 16 which are in each case embodied so as to be L-shaped but in a stand-alone manner. Two further functional elements 5 which have a reversed form-fit contour, each in the form of a rebate 13, are assigned to said two legs 16 and fixed thereon. Said further functional elements 5 are pushed below the respective L-shaped leg 16, so to speak, such that said L-shaped leg by way of the transverse leg thereof engages in the rebate 13. A form-fit is provided there. In order for the functional elements 5 to be additionally fixed, an input of energy has taken place in the region of said two lateral functional elements 5 such that corresponding materially integral connections 15 are configured there, just like a corresponding materially integral connection 14 between the fastening element 4 and the main panel 2 is of course also established by way of the input of energy. FIG. 4 serves as a schematic illustration showing that, apart from a combined bond of a materially integral bond and a form-fitting bond, only a purely form-fitting bond can also be provided.

Furthermore shown here are through holes 25 which are configured on the fastening element. The bearing face between the fastening element 4 and the main panel 2 is somewhat reduced by way of said through holes 25 such that the flexibility or elasticity of the main panel 2 is somewhat less affected by the welding of the fastening element 4, this being particularly expedient when the fastening element 4 per se is a flexible element.

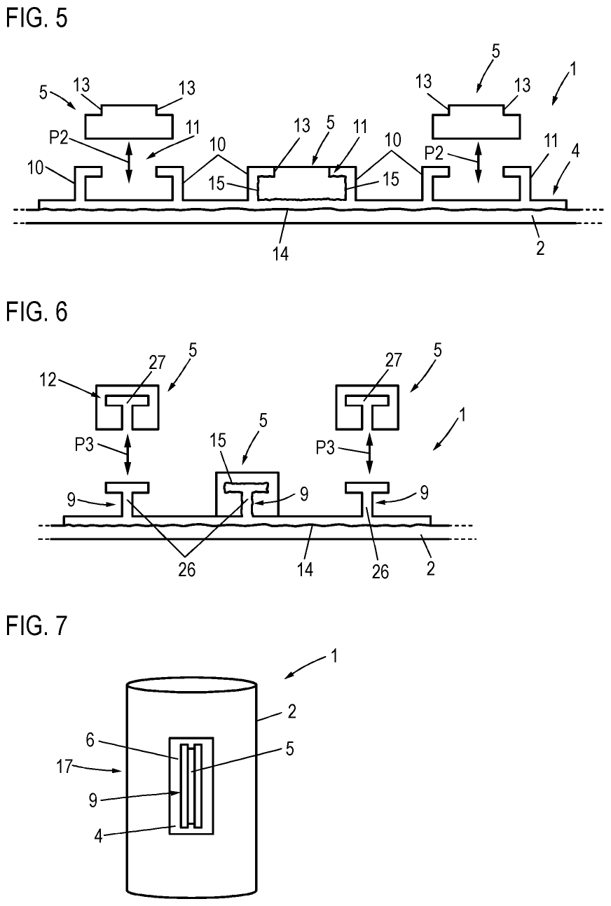

FIG. 5 shows a third exemplary embodiment of a textile part 1 according to the invention. Provided again are the main panel 2 as well as the fastening element 4 on which three receptacles for three functional elements 5 are again provided, said functional elements 5 having in each case two L-shaped legs 10 which define in each case one groove 11 undercut on both sides. The three functional elements 5 (of course, only two or more than three functional elements 5 may also be provided, as in the other examples) have in each case one shape-compatible T-shaped geometry having in each case two rebates 13 such that said functional elements 5 can engage in a form-fitting manner in the respective groove 11, as is shown by way of the central functional element 5.

In the exemplary embodiment shown, besides the materially integral welded connection 14 between the fastening element 4 and the main panel 2, a materially integral connection 15 is again implemented between the central functional element 5 and the fastening element 4, or the legs 10 of the latter, respectively, as is shown there by way of the serrated lines. The two functional elements 5 provided in a neighboring manner are able to be inserted into the corresponding grooves 11 but can also be retrieved therefrom, as is illustrated by the double arrows P2. This means that the user has the potential choice of inserting one neighboring functional element or both neighboring functional elements in addition to the centric functional element 5, and on account thereof of varying the properties of the textile part 1.

If the functional elements 5 are stabilizing stiffeners, for example, the textile part thus has first basic stabilizing by way of the centric stabilizing stiffener. Said first basic stabilizing can be enhanced when one additional functional element 5 or both additional functional elements 5, thus stabilizing stiffeners, are snap-fitted. Said additional functional elements 5 can also be retrieved again when required, since only a form-fit is provided.

The corresponding functional elements 5, or stabilizing stiffeners, respectively, herein can run so as to be mutually parallel or at a mutual angle; said functional elements 5 can have dissimilar lengths or else dissimilar mechanical stiffnesses, etc., such that a large bandwidth of variation is provided.

FIG. 6 shows a design embodiment of a textile part 1 having a main panel 2, again preferably a textile knitted panel, a fastening element 4, as well as three functional elements 5 here too. Corresponding form-fit contours 9, 12 are configured on the fastening element 4 as well as on the functional elements 5 here too. As opposed to the undercut grooves on the fastening element 4, T-shaped form-fit contours 9 which as elongate T-shaped splints extend along the fastening element 4, for example, are configured here on the fastening element 4. The form-fit contour 9 is embodied as a T-shaped protrusion 26.

Each functional element 5 has a corresponding form-fit contour 12 by way of which said functional element 5 can be snap-fitted to the T-shaped form-fit contours 9, as is shown in the case of the central functional element 5. A corresponding materially integral connection 15 is again also established on said central functional element 5, as is indicated by the serrated line, that is to say that, besides the form-fit, a non-releasable materially integral connection is additionally also provided. The form-fit contour 12 is as a T-shaped groove 27 undercut on both sides.

The two neighboring functional elements 5 however can be disposed or removed depending on the requirement, see the double arrows P3, to which end said functional elements 5 are only to be snap-fitted onto the corresponding T-shaped form-fit contours 9.

In a manner similar to that according to FIG. 4, a large bandwidth of variation in terms of the basic design of the properties of the textile part, in particular with a view to the therapeutic task to be met exists only in the case of this exemplary embodiment. This herein can be functional elements of dissimilar stiffnesses or functional elements having identical mechanical properties. Said functional elements can differ in terms of the length thereof, just like said functional elements can run so as to be mutually parallel or at a mutual angle, etc. The form-fitting connection 14 of the fastening element 5 toward the main panel 2 is of course also provided here, as is illustrated by the serrated line, said materially integral connection 14 being provided in all variants of the invention.

FIG. 7 shows a first specific exemplary implementation of a textile part 1 in the form of a bandage 17. Shown is the main panel 2, preferably again a textile main panel in the form of a knitted fabric, a woven fabric, or a warp-knitted fabric. A compressive knitted fabric in the form of a circular-knitted or flat-knitted fabric is preferably used. Furthermore shown is the fastening element 4 which is welded in a materially integral manner to the external side 3 of the main panel 2, as well as a functional element 5 which here is received on the form-fit contour 9 of the fastening element 4, thus is snap-fitted in the groove undercut on both sides. Said functional element 5 is fixed in a form-fitting manner in said groove, wherein the fixing here, since the form-fit contour 9 is open on both sides, is a fixing by clamping. This assembly thus corresponds to that as shown in FIGS. 1 to 3.

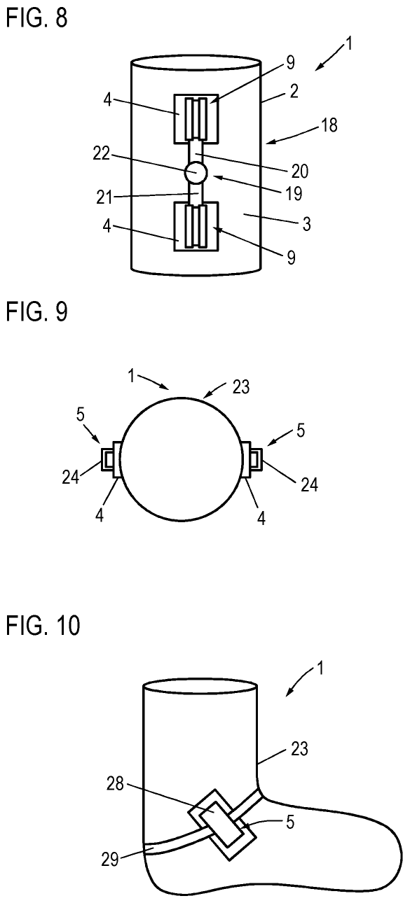

FIG. 8 shows a textile part 1 in the form of an orthotic 18. Two fastening elements 4 are fixed in a materially integral manner to said orthotic 18, a common functional element 5, here in the form of a joint 19, being disposed on said fastening elements 4. The joint 19 is composed of two articulated stiffeners 20, 21 which by way of an articulated connection 22 are pivotally connected to one another. Each articulated leg 20, 21 is received in a form-fitting manner in a corresponding form-fit contour 9 which again corresponds to that according to FIGS. 1 to 3, for example, and is optionally also fixed in a materially integral manner in said form-fit contour 9.

FIG. 9 finally shows a textile part 1 in the form of a stocking 23, preferably a compression stocking. One fastening element 4 is in each case disposed on both sides on the main panel 2 here too, and the functional element 5, here in the form of a handle 24 to be manually gripped, is preferably disposed in a form-fitting and materially integral manner on said fastening element 4, said handle 24 serving as dressing aids for pulling up the stocking 23. Since a relatively great force is to be transmitted here, the handles 24 are fixed in a form-fitting and materially integral manner.

FIG. 10 finally shows a textile part 1 in the form of a stocking 23 on which the functional element 5 in the form of a belt-guiding component 28 having a through hole is disposed by means of the fastening element 4, a belt 29 by way of which restraining can take place being guided through said belt-guiding component 28.

The invention is not limited to the exemplary embodiment shown, neither in terms of the design embodiment of the form-fit contours nor the disposal and number of the latter, and neither in terms of the specific variants of implementation in the form of a bandage, an orthotic, or a stocking. Rather, other design embodiments are also conceivable.

While specific embodiments of the invention have been shown and described in detail to illustrate the inventive principles, it will be understood that the invention may be embodied otherwise without departing from such principles.

I claim:

1. A textile part, in particular a textile orthopedic aid, comprising a textile main panel as well as at least one functional element from plastics material which is disposed on the main panel, wherein the functional element is connected either in a form-fitting or a form-fitting and materially integral manner to at least one fastening element, and the fastening element is connected in a materially integral manner to the main panel.

2. The textile part according to claim 1, wherein the fastening element is flexible or rigid.

3. The textile part according to claim 1, wherein the fastening element is a plastics-material element or has a carrier having a plastics-material coating on one side or both sides, or in that a fused intermediate tier which establishes the materially integral connection is provided between the fastening element and the main panel.

4. The textile part according to claim 1, wherein the functional element and/or the fastening element or the plastics-material coating or the intermediate tier are/is composed of polyamide, polyvinylchloride, polyurethane, polyethylene, or a thermoplastic elastomer, in particular a urethane-based thermoplastic elastomer.

5. The textile part according to claim 1, wherein a plurality of functional elements are disposed either in a form-fitting or a form-fitting and materially integral manner on a common fastening element.

6. The textile part according to claim 1, wherein a functional element which is composed of a plurality of parts that are able to move relative to one another is provided, wherein one part is connected either in a form-fitting or a form-fitting and materially integral manner to a first fastening element, and one further part is connected either in a form-fitting or a form-fitting and materially integral manner to a second fastening element, and both fastening elements are fastened in a materially integral manner to the main panel joint.

7. The textile part according to claim 1, wherein at least one groove which is undercut on one side or both sides and in which the functional element is received is provided on the fastening element.

8. The textile part according to claim 1, wherein at least one L-shaped or T-shaped protrusion which is received in a groove that is undercut on one side or both sides on the functional element is configured on the fastening element.

9. The textile part according to claim 1, wherein one through hole or a plurality of through holes is/are provided on the fastening element.

10. The textile part according to claim 1, wherein the functional element or elements and the fastening element or elements are joined by a plastic coating method, in particular a two-component plastic coating method.

11. The textile part according to claim 1, wherein the main panel is an in particular compressive knitted panel.

12. The textile part according to claim 1, wherein said textile part is a bandage, an orthotic, or a stocking, in particular a compression stocking, on which an elongate or curved rod-type functional element, a joint-type functional element, a functional element configured as a belt loop, a functional element configured as a traction aid that is able to be manually gripped, or a functional element configured as a pad is provided.

13. A method for producing a textile part according to claim 1, wherein at least one functional element which is connected either in a form-fitting or a form-fitting and materially integral manner to at least one fastening element is disposed on a textile main panel, whereupon a materially integral connection between the fastening element and the main panel is established by an input of energy.

14. The method according to claim 13, wherein a materially integral connection between the functional element and the fastening element is also established by the input of energy.

15. The method according to claim 13, wherein the fastening element, or the fastening element and the functional element, or an intermediate tier disposed between the fastening element and the main panel, is/are locally fused in order for the connection to be established.
